(19) United States
(12) Patent Application Publication
(10) Pub. No.: US 2026/0269058 A1
(43) Pub. Date: Sep. 10, 2026

(54) Storage Medium, Information Processing Method, and Information Processing Apparatus

(71) Applicant: Osaka University, Suita-shi, Osaka (JP)

(72) Inventors: Ken Nakata, Suita-shi, Osaka (JP); Issei Ogasawara, Suita-shi, Osaka (JP); Shoji Konda, Suita-shi, Osaka (JP); Yoshihiro Marutani, Suita-shi, Osaka (JP)

(21) Appl. No.: 18/846,987

(22) PCT Filed: Jan. 12, 2023
(86) PCT No.: PCT/JP2023/000588

§ 371 Date: Sep. 13, 2024

(30) Foreign Application Priority Data
Mar. 18, 2022 (JP) 2022-044253

(51) Int. Cl. G16H 40/67

(52) U.S. Cl. CPC G16H 40/67

(57) ABSTRACT

Provided are a program and the like capable of accurately evaluating the activity intensity of physical activity. A computer acquires acceleration data detected by an acceleration sensor attached to a subject in exercise. In addition, the computer counts the frequency of each acceleration based on the acquired acceleration data, and generates a frequency distribution for each acceleration.

Then, the computer specifies a mixed normal distribution model that expresses a frequency distribution for each acceleration generated.

### REFERENCE TO RELATED APPLICATIONS

This application is the national phase under 35 U.S. C. § 371 of PCT International Application No. PCT/JP2023/000588 which has an International filing date of Jan. 12, 2023 and designated the United States of America.

### BACKGROUND ART

With the widespread use of wearable sensors, it is possible to monitor the activity intensity of physical activities in daily life, work, sports, exercise, rehabilitation, and the like. For example, Japanese Patent Laid-Open Publication No. 2022-19140 discloses a technique for measuring user's heart rate data and acceleration data using a sensor device attached to the user's chest and calculating the user's exercise intensity based on the measurement results.

### SUMMARY

In the process of calculating the user's activity intensity, for example, an average value of acceleration in an analysis target period is often used. Japanese Patent Laid-Open Publication No. 2022-19140 discloses that metabolic equivalents (METs), which indicate exercise intensity, are calculated using an average value of acceleration in a predetermined period. However, some physical activities, such as walking and running, involve continuous movements of almost constant activity intensity, while other physical activities, such as ball sports, involve a mixture of movements of various activity intensities, ranging from being nearly stationary to sprinting at full speed. In such physical activities that involve a mixture of movements with different activity intensities, for example, the average value of acceleration may be a value of medium intensity, making it difficult to appropriately express the characteristics of the activity intensity in the physical activity. Therefore, it may not be appropriate to evaluate the user's activity intensity based on the average value of acceleration in the analysis target period.

The present disclosure has been made in view of such circumstances, and an object thereof is to provide a storage medium and the like capable of accurately evaluating the activity intensity of physical activity.

A non-transitory computer-readable storage medium according to one aspect of the present disclosure stores a program causing a computer to execute processing of: acquiring acceleration data detected by an acceleration sensor attached to a subject in exercise; counting a frequency of each acceleration based on the acceleration data; and specifying a mixed normal distribution model that expresses a frequency distribution for each acceleration.

According to one aspect of the present disclosure, it is possible to evaluate the activity intensity of physical activity with high accuracy.

The above and further objects and features will more fully be apparent from the following detailed description with accompanying drawings.

### BRIEF DESCRIPTION OF DRAWINGS

FIG. 1 is an explanatory diagram showing an example of the configuration of an information processing system.

FIG. 2 is a block diagram showing an example of the configuration of an information processing system.

FIG. 3 is a flowchart showing an example of the procedure of an activity intensity determination process.

FIG. 4A is an explanatory diagram of the activity intensity determination process.

FIG. 4B is an explanatory diagram of the activity intensity determination process.

FIG. 5 is an explanatory diagram showing a screen example.

FIG. 6A is an explanatory diagram showing a modified example of an exercise evaluation screen.

FIG. 6B is an explanatory diagram showing a modified example of the exercise evaluation screen.

FIG. 7 is an explanatory diagram showing a modified example of the exercise evaluation screen.

FIG. 8 is a flowchart showing an example of the procedure of an in-play time determination process.

FIG. 9 is an explanatory diagram of the in-play time determination process.

FIG. 10 is a flowchart showing an example of the procedure of an activity intensity determination process according to Embodiment 2.

FIG. 11 is an explanatory diagram showing a modified example of the exercise evaluation screen.

FIG. 12A is an explanatory diagram of the process of evaluating the physical activity content based on activity intensity on the high intensity side.

FIG. 12B is an explanatory diagram of the process of evaluating the physical activity content based on activity intensity on the high intensity side.

FIG. 13 is a flowchart showing an example of the procedure of an activity intensity determination process according to Embodiment 3.

FIG. 14 is an explanatory diagram showing a screen example.

FIG. 15 is an explanatory diagram showing a modified example of the exercise evaluation screen.

### DESCRIPTION

Hereinafter, a program, an information processing method, and an information processing apparatus according to the present disclosure will be described with reference to the diagrams showing embodiments thereof. In each of the following embodiments, a configuration in which the characteristics of the activity intensity applied to a person (human being) when the person exercises are determined will be described as an example. However, the determination target in each embodiment is not limited to persons and may be, for example, livestock, laboratory animals, and working animals.

### Embodiment 1

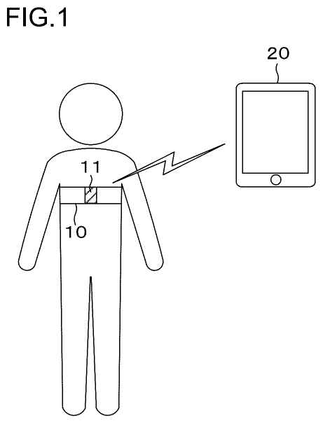

An information processing system for determining the characteristics of the activity intensity applied to a user (person) during exercise will be described. FIG. 1 is an explanatory diagram showing an example of the configuration of an information processing system. The information processing system according to the present embodiment includes a wearable device 10 attached to the body of a user (subject) performing exercise and an information processing apparatus 20, and the wearable device 10 and the information processing apparatus 20 are configured to perform, for example, wireless communication with each other. The exercise performed by the user in the present embodiment includes all physical activities such as rehabilitation and labor, as well as various sports and exercises including walking and running.

The wearable device 10 is formed, for example, in a belt shape, and is configured to be attached to the chest of the user by attaching hook-and-loop fasteners provided at both ends. An acceleration sensor 11 is provided in the wearable device 10, and the acceleration sensor 11 is placed on the user's sternum or upper back when the user wears the wearable device 10 on his or her chest. In addition, the wearable device 10 may be configured to be attached to an attaching belt for attaching a mobile terminal such as a smartphone, a tablet terminal, or a portable game console having an acceleration sensor to the user's chest. In addition, the wearable device 10 may be configured such that the acceleration sensor 11 is attached to the chest or upper back of a garment such as an undershirt, or may be configured by attaching the acceleration sensor 11 or a mobile terminal having an acceleration sensor to a pocket provided on the chest or upper back of the garment. The acceleration sensor 11 is preferably attached to a position close to the center (trunk) of the body of the user (human being) in order to appropriately detect impacts received by the user during exercise. Therefore, in the present embodiment, the acceleration sensor 11 is configured to be attached to the user's chest, but is not limited to this configuration. For example, the acceleration sensor 11 may be attached to an appropriate position on the user's torso, such as the waist.

The information processing apparatus 20 can perform various kinds of information processing and transmit and receive information, and is configured by, for example, a tablet terminal, a smartphone, a portable game console, or a personal computer. In addition, the information processing apparatus 20 can be configured using various apparatuses having a communication unit that communicates with the wearable device 10. In the present embodiment, the wearable device 10 and the information processing apparatus 20 are configured to perform wireless communication with each other, but may be configured to be wired to each other through a cable such as a USB (Universal Serial Bus) cable. In addition, when a mobile terminal having an acceleration sensor is used as the wearable device 10, the wearable device 10 (acceleration sensor 11) and the information processing apparatus 20 may be a mobile terminal configured as one unit. In addition, when the wearable device 10 is configured to be able to write and read data to and from a portable storage medium such as an SD (Secure Digital) card or a microSD card, data may be transmitted from the wearable device 10 to the information processing apparatus 20 through the portable storage medium. The information processing apparatus 20 according to the present embodiment receives acceleration data (acceleration signal) that is the detection result of the acceleration sensor 11 provided in the wearable device 10, and determines the activity intensity applied to the user during exercise based on the acceleration data.

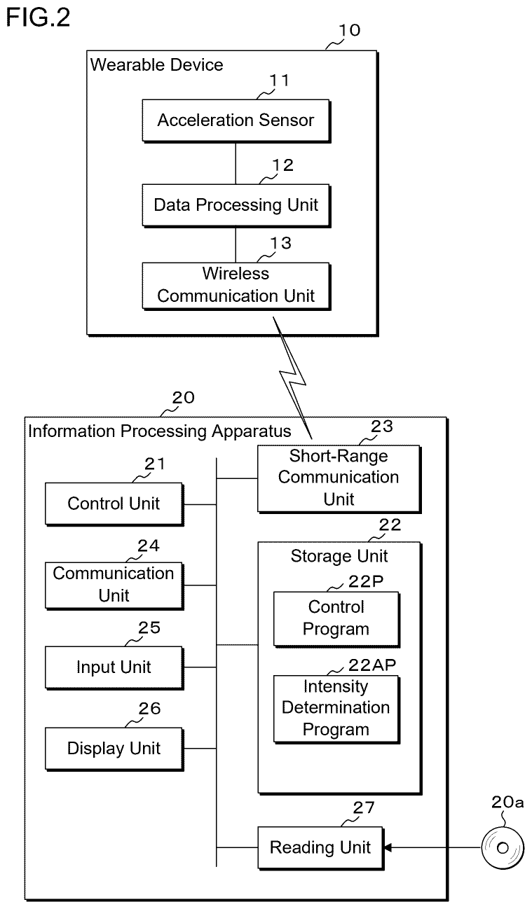

FIG. 2 is a block diagram showing an example of the configuration of an information processing system. The wearable device 10 includes an acceleration sensor 11, a data processing unit 12, a wireless communication unit 13, and the like. The acceleration sensor 11 is a three-axis acceleration sensor, and detects acceleration in three directions, up and down, left and right, and front and back, according to the movement of the user wearing the wearable device 10. As the acceleration sensor 11, a sensor that measures acceleration at a sampling frequency of, for example, about 200 Hz to 1000 Hz is used. In the present embodiment, since the wearable device 10 is attached when the user performs physical activity, the acceleration sensor 11 detects acceleration applied to the user's body according to the user's physical activity (movement).

The data processing unit 12 includes an arithmetic processor such as a CPU (Central Processing Unit) or an MPU (Micro-Processing Unit), a memory, a clock, and the like. The data processing unit 12 converts the acceleration detected by the acceleration sensor 11 into digital data using an arithmetic processor, and stores or accumulates in a memory the acceleration data including the converted acceleration and information indicating the measurement point in time of the acceleration. The information indicating the measurement point in time of the acceleration may be information indicating the elapsed time from the start of the measurement of the acceleration, or may be date and time information indicated by a clock. When the wearable device 10 can write data to a portable storage medium, the acceleration data processed by the data processing unit 12 may be stored in the portable storage medium.

The wireless communication unit 13 transmits the acceleration data, which has been digitized by the data processing unit 12 and stored in the memory, to the information processing apparatus 20 through wireless communication. The wireless communication unit 13 transmits the acceleration data by wireless communication conforming to, for example, Bluetooth®. The wireless communication unit 13 may transmit the acceleration data stored in the memory by the data processing unit 12 to the information processing apparatus 20 in real time, or may transmit all pieces of acceleration data detected during a predetermined period of time to the information processing apparatus 20 after all the pieces of acceleration data are accumulated in the memory.

The information processing apparatus 20 includes a control unit 21, a storage unit 22, a short-range communication unit 23, a communication unit 24, an input unit 25, a display unit 26, a reading unit 27, and the like, and these units are connected to each other through a bus. The control unit 21 includes one or more processors such as a CPU, an MPU, or a GPU (Graphics Processing Unit). The control unit 21 executes a control program 22P stored in the storage unit 22 as appropriate to execute processing to be performed by the information processing apparatus 20.

The storage unit 22 includes a RAM, a flash memory, a hard disk, an SSD (Solid State Drive), and the like. The storage unit 22 stores in advance the control program 22P executed by the control unit 21 and various kinds of data required for the execution of the control program 22P. In addition, the storage unit 22 temporarily stores data and the like that are generated when the control unit 21 executes the control program 22P. In addition, the storage unit 22 stores an intensity determination program 22AP to realize a process of determining the activity intensity applied to the user based on the acceleration data received from the wearable device 10. The control program 22P and the intensity determination program 22AP (program products) and various kinds of data stored in the storage unit 22 may be written during the manufacturing stage of the information processing apparatus 20, or may be downloaded from other devices through the communication unit 24 by the control unit 21 and stored in the storage unit 22.

The short-range communication unit 23 is a communication device that performs wireless communication with the wearable device 10. The short-range communication unit 23 performs wireless communication conforming to, for example, Bluetooth. The communication unit 24 has an interface for connecting to a network, such as the Internet, by wired or wireless communication, and transmits and receives information to and from other devices through the network.

The input unit 25 receives an operation input by the user who operates the information processing apparatus 20, and transmits a control signal corresponding to the operation content to the control unit 21. The display unit 26 is a liquid crystal display, an organic EL display, and the like, and displays various kinds of information according to instructions from the control unit 21. A part of the input unit 25 and the display unit 26 may be a touch panel configured as one unit.

The reading unit 27 reads information stored in a portable storage medium 20a such as a CD (Compact Disc)-ROM, a USB (Universal Serial Bus) memory, an SD card, a microSD card, or a Compact Flash®. The programs 22P and 22AP and data stored in the storage unit 22 may be read from the portable storage medium 20a through the reading unit 27 by the control unit 21 and stored in the storage unit 22.

The information processing apparatus 20 according to the present embodiment acquires acceleration data detected by the acceleration sensor 11 provided in the wearable device 10 during the user's physical activity (exercise), and calculates the activity intensity (degree of impact) applied to the user by the physical activity based on the acceleration data. Then, the information processing apparatus 20 performs a process of presenting the activity intensity applied to the user to the user or the like.

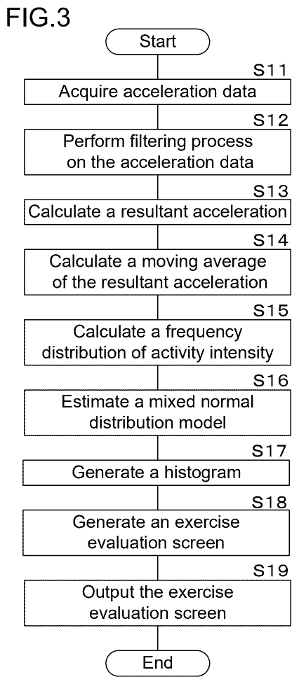

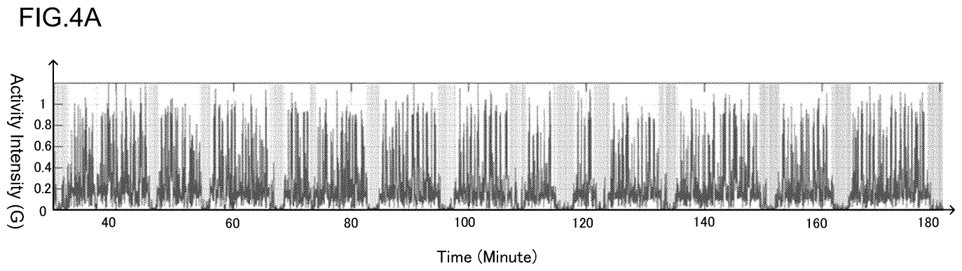

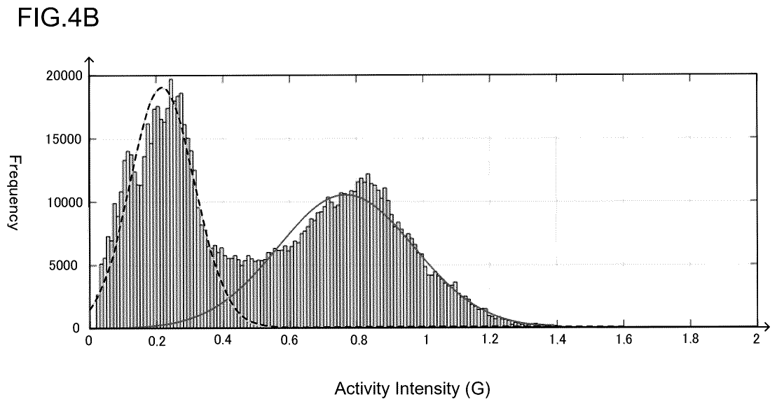

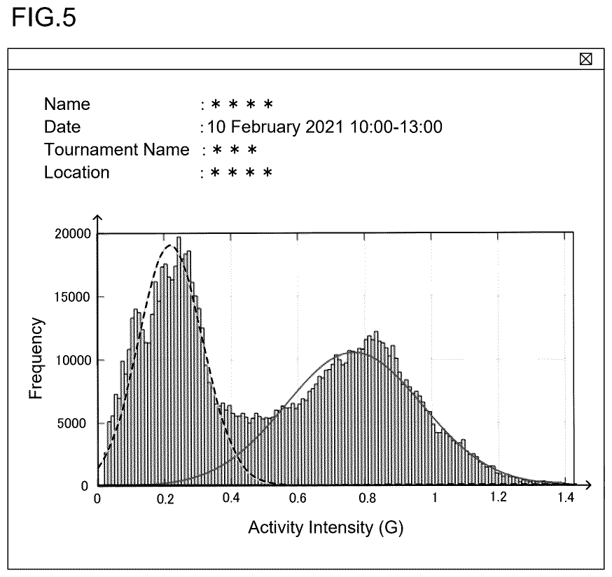

Hereinafter, a process will be described in which the information processing apparatus 20 determines the activity intensity (degree of impact) applied to the user based on the acceleration data detected by the acceleration sensor 11 attached to the user during physical activity in the information processing system according to the present embodiment. FIG. 3 is a flowchart showing an example of the procedure of the activity intensity determination process, FIGS. 4A and 4B are explanatory diagrams of the activity intensity determination process, and FIG. 5 is an explanatory diagram showing an example of a screen. The following process is performed by the control unit 21 according to the control program 22P and the intensity determination program 22AP stored in the storage unit 22 of the information processing apparatus 20. In the following process, the user wears the wearable device 10 on his or her chest and performs physical activity, and the acceleration sensor 11 measures acceleration.

The control unit 21 (acquisition unit) of the information processing apparatus 20 acquires acceleration data, which is generated from acceleration periodically detected by the acceleration sensor 11, from the wearable device 10 attached to the user (subject) during the physical activity (S11). Since the acceleration sensor 11 measures acceleration at a sampling frequency of, for example, about 200 Hz to 1000 Hz, the control unit 21 acquires, as acceleration data, a waveform signal obtained by measuring the acceleration approximately every 1 ms to 5 ms. The wearable device 10 may transmit acceleration data, which is detected by the acceleration sensor 11 during the user's physical activity, to the information processing apparatus 20 in real time, or may transmit all pieces of acceleration data, which are detected by the acceleration sensor 11 during the physical activity, to the information processing apparatus 20 after the end of the user's physical activity. In addition, the wearable device 10 may transmit acceleration data detected by the acceleration sensor 11 to the information processing apparatus 20 in response to a request from the information processing apparatus 20. The method of transmitting acceleration data from the wearable device 10 to the information processing apparatus 20 is not limited to wireless communication, and may be wired communication through a cable or may be through a portable storage medium. In addition, as for the physical activity, for example, one match in sports may be a series of physical activities, or one game, one set, or a first or second half of one match may be a series of physical activities. Alternatively, each sport practice menu may be a series of physical activities. In addition, in the case of exercise, rehabilitation, work, and the like, the duration of the activity may be a series of physical activities.

The control unit 21 performs a filtering process on the acquired acceleration data to extract predetermined frequency components (S12). For example, the control unit 21 performs a filtering process using a band pass filter that extracts frequency components of 0.5 Hz to 5 Hz. Therefore, since noise components included in the acceleration data are removed, it is possible to extract data on the acceleration applied to the user due to physical activity. In addition, since the acceleration sensor 11 is a three-axis acceleration sensor, the control unit 21 acquires acceleration data in each of the three directions and performs a filtering process on each of the pieces of acceleration data in the three directions.

Then, the control unit 21 calculates a resultant acceleration by combining the accelerations in the three directions based on the filtered acceleration data in the three directions (S13). The resultant acceleration is calculated by taking the square root of the sum of the squares of the accelerations in the three directions. Then, the control unit 21 calculates a moving average of the resultant acceleration (S14). Here, the control unit 21 calculates a simple moving average every predetermined period of time, for example, about 0.5 seconds to 1.0 seconds. Specifically, for a certain time, the control unit 21 calculates an average value of the resultant acceleration from that time to before a predetermined time, and stores the calculated average value in the storage unit 22 in association with that time. The control unit 21 calculates an average value of the resultant acceleration during a predetermined period of time every acceleration measurement timing, and calculates a moving average of the resultant acceleration for all pieces of acceleration data measured during the user's physical activity.

FIG. 4A shows an example of a time-series change in the moving average of the resultant acceleration, specifically, a time series change in the moving average of the resultant acceleration calculated based on acceleration data measured during a tennis match. In the waveform shown in FIG. 4A, the horizontal axis indicates elapsed time from the start of the user's physical activity (here, a tennis match), and the vertical axis indicates the moving average of the resultant acceleration (activity intensity). In addition, in the following description, the moving average of the resultant acceleration is referred to as activity intensity, and the content of the user's physical activity is evaluated based on this activity intensity. In the present embodiment, the moving average of the resultant acceleration is used as an index of activity intensity, but the present invention is not limited to this configuration. For example, the average value of the resultant acceleration for each predetermined period of time may be used as an index of activity intensity.

Then, the control unit 21 calculates a frequency distribution of each activity intensity based on the calculated activity intensity (moving average of resultant acceleration) (S15). For example, the control unit 21 (counting unit) calculates a frequency distribution by counting the frequency of the activity intensity every 0.01 G. FIG. 4B shows a frequency distribution of activity intensity calculated from the time-series data of the activity intensity shown in FIG. 4A. In the distribution shown in FIG. 4B, the horizontal axis indicates activity intensity, and the vertical axis indicates frequency. The distribution in FIG. 4B shows that high-intensity activities (movements) with an activity intensity of about 0.8 and low-intensity activities (movements) with an activity intensity of about 0.2 are mixed in a tennis match. In such physical activity including high-intensity movements and low-intensity movements, it is difficult to express the characteristics of the physical activity by, for example, the average value of all activity intensities measured during the physical activity. Therefore, in the present embodiment, the characteristics of the physical activity are evaluated by expressing the frequency distribution of activity intensity using a mixed normal distribution model that mixes two normal distributions, a normal distribution on the high intensity side and a normal distribution on the low intensity side.

Therefore, the control unit 21 (specifying unit) estimates a mixed normal distribution model that approximates the frequency distribution of the activity intensity (S16). The mixed normal distribution model can be estimated by a known fitting process such as the EM algorithm (Expectation-Maximization algorithm) or variational Bayes, and parameters of the mixed normal distribution model that maximizes the calculated likelihood function are estimated. In the EM algorithm, based on the frequency distribution of activity intensity and the number of normal distributions to be mixed (two in this case), the mean and standard deviation representing the normal distribution on the high intensity side, the mean and standard deviation representing the normal distribution on the low intensity side, and the mixture ratio of the two normal distributions are estimated (calculated). Therefore, as the estimation result of the mixed normal distribution model, the control unit 21 acquires parameters including the mean and standard deviation in the normal distribution on the high intensity side, the mean and standard deviation in the normal distribution on the low intensity side, and the mixture ratio of the two normal distributions.

The control unit 21 generates a histogram showing the frequency distribution of the activity intensity calculated in step S15 (S17). For example, the control unit 21 generates a histogram shown in FIG. 4B. Then, the control unit 21 generates an exercise evaluation screen for presenting the mixed normal distribution model estimated in step S16 to notify of information regarding the user's exercise content (S18). For example, as shown in FIG. 5, the control unit 21 generates a screen that displays a curve showing a mixed normal distribution model so as to be superimposed on a histogram. Specifically, the control unit 21 generates a curve showing the normal distribution on the high intensity side based on the mean and standard deviation and the mixing ratio of the normal distribution on the high intensity side, which are obtained by the estimation process of the mixed normal distribution model, generates a curve showing the normal distribution on the low intensity side based on the mean and standard deviation and the mixing ratio of the normal distribution on the low intensity side, and displays the two curves showing the normal distributions so as to be superimposed on a histogram. In the example shown in FIG. 5, the curve showing the normal distribution on the low intensity side is shown by a dashed line, and the curve showing the normal distribution on the high intensity side is shown by a solid line. In addition, the screen shown in FIG. 5 displays information about the user, such as the name of the user (for example, a player) to be evaluated, the date and time of the physical activity, the name of the tournament and its location, together with a histogram with curves showing two normal distributions. In addition, the information about the user may be input through the input unit 25, or may be input in advance and stored in the storage unit 22.

The control unit 21 outputs the generated exercise evaluation screen (S19). For example, the control unit 21 displays an exercise evaluation screen shown in FIG. 5 on the display unit 26. In addition, the control unit 21 may transmit the exercise evaluation screen to a predetermined terminal, or may transmit the exercise evaluation screen to a communicable printer for printing. In this manner, it is possible to present to the user the distribution characteristics of the activity intensity received by the user during the user's physical activity by using the mixed normal distribution model, and it is possible to output information regarding the physical activity performed by the user. With such a screen, in physical activity in which high-intensity movements and low-intensity movements are mixed, the frequency (amount of exercise) of each intensity (high intensity side and low intensity side) can be grasped. In addition, by displaying the curve showing the mixed normal distribution model so as to be superimposed on a histogram showing the frequency distribution of activity intensity, it is easy to intuitively grasp the amount of exercise on the high intensity side and the amount of exercise on the low intensity side. Therefore, the information processing apparatus 20 can output information regarding the user's exercise based on the mixed normal distribution model, making it possible to evaluate the quality and quantity of the exercise, for example, whether or not the user can perform practice (movements) with high activity intensity.

By expressing the frequency distribution of activity intensity using the mixed normal distribution model as described above, the activity intensity in a series of physical activities can be classified into a distribution of activity intensity in movements in a state close to a stationary state (distribution on the low intensity side) and a distribution of activity intensity in movements in a state close to a sprint (distribution on the high intensity side). Therefore, even in the case of physical activity in which movements of various activity intensities, ranging from a state close to a stationary state to sprinting are mixed, it is possible to divide the activity intensities into the high intensity side and the low intensity side and appropriately evaluate the activity content based on the respective distributions. Thus, since the activity content of each user (athlete, player, and the like) can be presented objectively, it is possible to support the improvement of the activity content of each user.

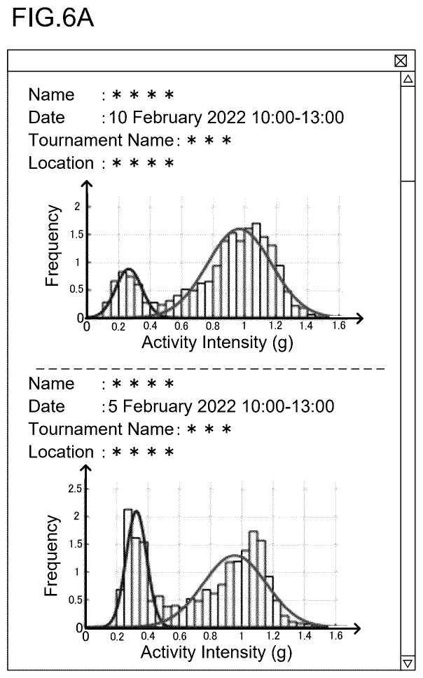

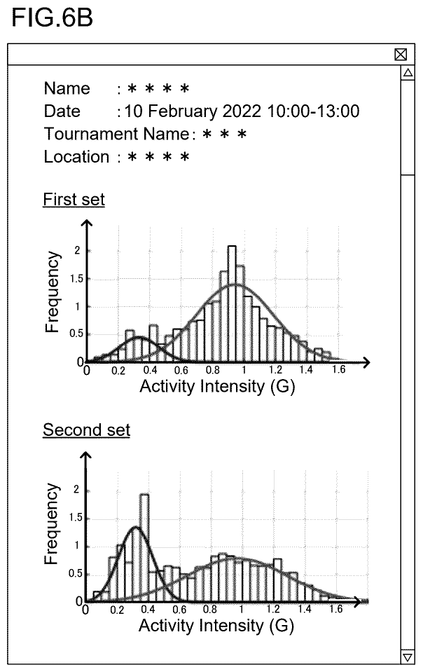

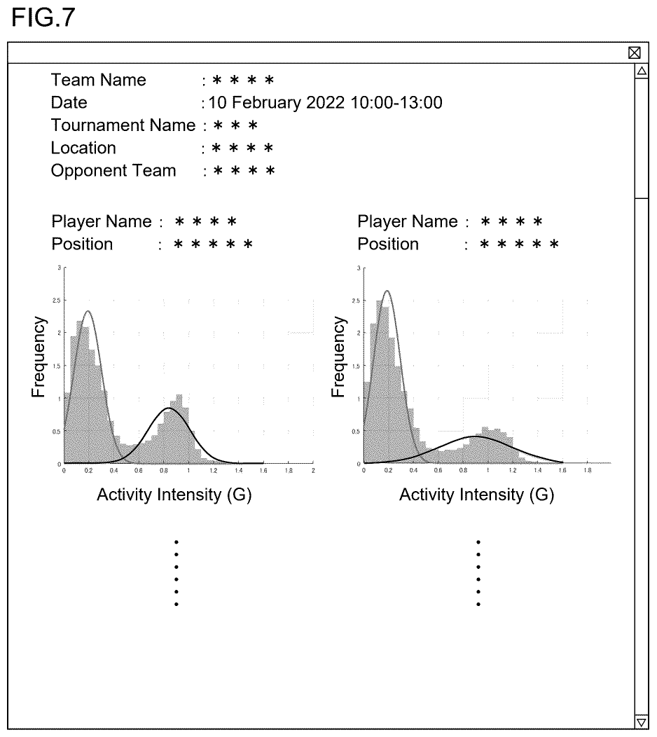

FIGS. 6A to 7 are explanatory diagrams showing modified examples of the exercise evaluation screen. In the present embodiment, for example, when data on the activity intensity in each user's past physical activity is stored in the storage unit 22 or other storage devices, the data may be presented together with data on the activity intensity in the current physical activity. In this case, in step S18 in the process shown in FIG. 3, the control unit 21 may generate an exercise evaluation screen that displays a graph in which a histogram generated from acceleration data obtained in the current physical activity is superimposed on a curve showing a mixed normal distribution model (two normal distributions) that approximates the histogram and a graph in which a histogram of past physical activity is superimposed on a curve showing a mixed normal distribution model (two normal distributions) that approximates the histogram, as shown in FIG. 6A. By presenting the screen shown in FIG. 6A, the distribution of activity intensity for each physical activity of one user can be presented side by side, so that it is possible to easily compare the distribution of activity intensity for each physical activity. For example, when high-intensity activity is continued, the peak value (average value) of the normal distribution on the high intensity side tends to move to the low intensity side, or the frequency of the peak value of the normal distribution on the high intensity side tends to decrease. Therefore, according to whether or not such a tendency is observed, it is possible to evaluate whether or not high-intensity activity can be continued. In addition, for example, when the frequency of peak values of the normal distribution on the low intensity side increases and the frequency of peak values of the normal distribution on the high intensity side decreases in physical activities that occur before and after in a chronological order, it can be determined that the high-intensity activity cannot be continued due to fatigue or the like. In addition, the past physical activity to be compared may be the most recent physical activity, the same competition as the current physical activity, physical activity of the same activity duration, and the like, and comparison with the activity intensity in any physical activity to be compared is possible.

In addition, for example, acceleration data measured during one tennis match shown in FIG. 4A may be divided into sets during one match, and activity intensity data for each set may be presented. In this case, after the processing of step S13 in the process shown in FIG. 3, the control unit 21 divides the calculated resultant acceleration for each set, and performs the processing of steps S14 to S18 based on the resultant acceleration for each set. Specifically, the control unit 21 calculates the moving average of the resultant acceleration for each set (S14), calculates the frequency distribution of the activity intensity (the moving average of the resultant acceleration) (S15), and estimates a mixed normal distribution model that approximates the frequency distribution of the activity intensity (S16). Then, the control unit 21 generates a histogram showing the frequency distribution of activity intensity for each set (S17), and generates an exercise evaluation screen displaying a graph in which a curve showing a mixed normal distribution model (two normal distributions) that approximates the generated histogram is superimposed on the generated histogram (S18). In this case, as shown in FIG. 6B, an exercise evaluation screen that presents the distribution of activity intensity in each set is generated. By presenting the screen shown in FIG. 6B, it is possible to compare the distribution of activity intensity of physical activity in each of a plurality of sets during one match, for example. In addition, the physical activity to be compared is not limited to the physical activity in each set during one match, but may be, for example, physical activities during respective periods of time into which a sports practice time is divided according to a practice menu, and comparison with the activity intensity in physical activity at any analysis target time is possible. In addition, by comparing the activity intensities at analysis target times before and after in a chronological order, it is possible to evaluate, for example, whether or not high-intensity physical activity can be performed continuously.

The screens shown in FIGS. 6A and 6B present the activity intensity of each physical activity of one user, but are not limited to this configuration. For example, for team competitions involving a plurality of players, data on the activity intensity of each player may be presented. In this case, in step S11 in the process shown in FIG. 3, the control unit 21 acquires acceleration data measured by the acceleration sensor 11 attached to each player and performs the processing of steps S12 to S17 based on the acceleration data acquired for each player. Specifically, the control unit 21 calculates a moving average of the resultant acceleration for each user (S14), calculates a frequency distribution of activity intensity (the moving average of the resultant acceleration) (S15), and estimates a mixed normal distribution model that approximates the frequency distribution of the activity intensity (S16). Then, the control unit 21 generates a histogram showing the frequency distribution of activity intensity for each user (S17), and generates an exercise evaluation screen displaying a graph in which a curve showing a mixed normal distribution model (two normal distributions) that approximates the generated histogram is superimposed on the generated histogram (S18). In this manner, since a graph is generated in which a curve showing a mixed normal distribution model (two normal distributions) that approximates a histogram showing the frequency distribution of activity intensity for each player is superimposed on the histogram, it is possible to generate an exercise evaluation screen displaying the histogram for each player. Therefore, as shown in FIG. 7, it is possible to present an exercise evaluation screen that presents the distribution of activity intensity for each user. By presenting the screen shown in FIG. 7, it is possible to compare the distribution of the activity intensity of the physical activity of each player during, for example, one match or practice time, enabling analysis of individual activity characteristics and activity characteristics for each position. Therefore, since the characteristics of the distribution of activity intensity for each player can be presented, it is possible to output information regarding the physical activity of each player.

In the present embodiment, the activity intensity of the physical activity performed by the user is evaluated based on the resultant acceleration obtained by combining accelerations in three directions measured using the three-axis acceleration sensor 11. Alternatively, instead of combining the accelerations in the three directions, the activity intensity in each direction may be evaluated by calculating the frequency distribution of the activity intensity for the acceleration in each direction. In addition, when the accelerations in the three directions are combined, weighting based on a weighting coefficient set for each of the three directions may be performed to calculate the resultant acceleration.

In the present embodiment, the process of determining the activity intensity applied to the user's body from the acceleration measured by the wearable device 10 attached to the user when performing physical activity is not limited to a configuration in which the information processing apparatus 20 performs the process locally. For example, a server that performs the above-described processes may be provided. In this case, the information processing apparatus 20 is configured to transmit the acceleration data received from the wearable device 10 to the server and acquire a frequency distribution of the user's activity intensity and a curve showing a mixed normal distribution model approximating the frequency distribution, generated by the server (specifically, the exercise evaluation screens shown in FIGS. 5 to 6B). Even in such a configuration, the same processing as in the present embodiment is possible, and the same effects can be obtained. In the case of providing a server as described above, a plurality of servers may be provided to perform distributed processing, or a server may be realized by a plurality of virtual machines provided in a single server or may be realized using a cloud server.

### Embodiment 2

An information processing system will be described that determines whether or not a user is playing (exercising or resting) based on acceleration data measured by the acceleration sensor 11 during the user's physical activity and determines the characteristics of the activity intensity applied to the user based on acceleration data measured in the time zone for which it is determined that the user is playing. For example, in ball sports, there are in-play times and out-of-play times, and the out-of-play times are considered to be times when the physical activity (play) that is the analysis target is stopped. Therefore, in the present embodiment, based on acceleration data measured during physical activity, it is determined whether the time is in-play time or out-of-play time, and the user's activity intensity is determined based on the acceleration data for the time determined to be in-play time. Since the information processing system according to the present embodiment is realized using the same apparatus as in the information processing system according to Embodiment 1 shown in FIG. 2, a description of the configuration will be omitted.

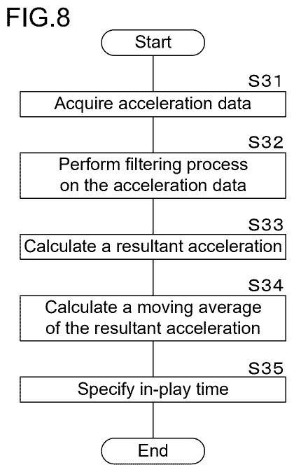

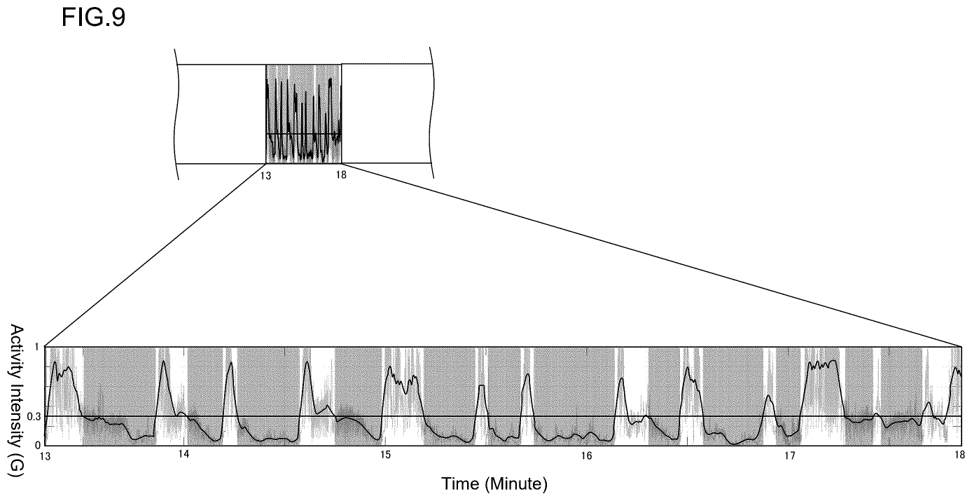

FIG. 8 is a flowchart showing an example of the procedure of an in-play time determination process, and FIG. 9 is an explanatory diagram of the in-play time determination process. The following process is performed by the control unit 21 according to the control program 22P and the intensity determination program 22AP stored in the storage unit 22 of the information processing apparatus 20.

In the information processing apparatus 20 according to the present embodiment, when performing the process of determining the in-play time and the out-of-play time based on the acceleration data measured by the acceleration sensor 11, the control unit 21 performs the same processing as steps S11 to S14 in the process shown in FIG. 3 (S31 to S34). As a result, the control unit 21 acquires acceleration data from the wearable device 10, performs a filtering process on the acquired acceleration data, calculates a resultant acceleration by combining the pieces of acceleration data in the three directions after the filtering process, and calculates a moving average of the calculated resultant acceleration. In addition, in step S34, the control unit 21 calculates a simple moving average every 60 seconds, for example. As a result, time-series data of the moving average (activity intensity) of the resultant acceleration is obtained as shown in FIG. 9.

The upper part of FIG. 9 shows a waveform indicating a part of the time-series data of the activity intensity, and the lower part of FIG. 9 shows the waveform in the upper part of FIG. 9 stretched in the time axis (horizontal axis) direction. In the wavelength shown in FIG. 9, the horizontal axis indicates elapsed time from the start of the user's physical activity, and the vertical axis indicates activity intensity. The waveform in the lower part of FIG. 9 shows that the time zone of high activity intensity and the time zone of low activity intensity are repeated. For example, in a tennis match, it is considered that the time zone of high activity intensity is the in-play time and the time zone of low activity intensity is the out-of-play time. Therefore, in the present embodiment, it is determined that a time zone, during which the activity intensity (for example, a simple moving average of the resultant acceleration every 60 seconds) is equal to or less than a predetermined value (0.3 in the example shown in FIG. 9), continues for a predetermined time (for example, 5 seconds) or more is not the in-play time, that is, is the out-of-play time.

Therefore, based on the moving average (activity intensity) of the resultant acceleration calculated in step S34, the control unit 21 specifies a time zone, during which the activity intensity equal to or less than the predetermined value continues for a predetermined time or more, as out-of-play time, and specifies a time zone other than the time zone determined as the out-of-play time as in-play time (S35). In the waveform shown in FIG. 9, time zones indicated in gray are out-of-play times, and time zones indicated in white are in-play times. The control unit 21 stores the specified time zone indicating in-play time in the storage unit 22, and ends the process.

Through the above-described process, the physical activity time in which acceleration is measured by the acceleration sensor 11 can be determined as in-play time and out-of-play time. By using the result of this determination, in the process of determining the activity intensity, it is possible to extract only the acceleration data measured during the in-play time and evaluate the activity intensity.

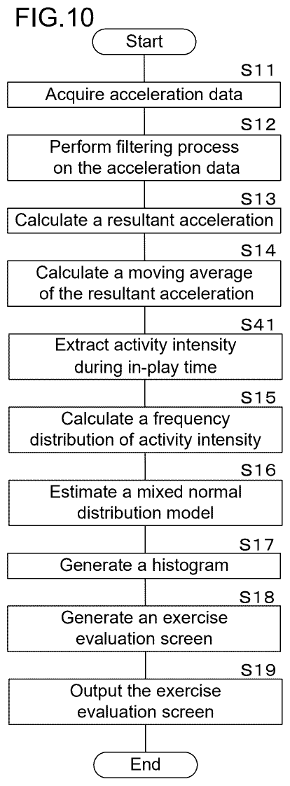

FIG. 10 is a flowchart showing an example of the procedure of an activity intensity determination process according to Embodiment 2. The process shown in FIG. 10 is obtained by adding step S41 between steps S14 and S15 in the process shown in FIG. 3. Explanation of the same steps as those in FIG. 3 will be omitted.

In the information processing apparatus 20 according to the present embodiment, the control unit 21 performs the same processing as steps S11 to S14 in FIG. 3. As a result, in the present embodiment as well, the information processing apparatus 20 performs a filtering process on the acceleration data acquired from the wearable device 10, calculates a resultant acceleration by combining the acceleration data in the three directions after the filtering process, and calculates a moving average (activity intensity) of the resultant acceleration. Thus, time-series changes in activity intensity (time-series data) shown in FIG. 4A are obtained.

From the calculated time-series changes in activity intensity, the control unit 21 extracts the activity intensity during the in-play time based on the in-play time specified by the in-play time determination process shown in FIG. 8 (S41). Then, the control unit 21 performs the processing of steps S15 to S19 based on the activity intensity during the in-play time. As a result, the control unit 21 calculates a frequency distribution of the activity intensity for the activity intensity during the in-play time, and estimates a mixed normal distribution model that approximates the calculated frequency distribution of the activity intensity. In addition, the control unit 21 generates a histogram showing the calculated frequency distribution of the activity intensity, and generates and outputs an exercise evaluation screen in which a curve showing the mixed normal distribution model that approximates the generated histogram is superimposed on the generated histogram. As a result, in the present embodiment as well, it is possible to present the exercise evaluation screens shown in FIGS. 5 to 7.

Through the above-described process, in the present embodiment, acceleration data in a time zone (for example, in-play time) during which the physical activity to be analyzed is performed can be extracted from the acceleration data measured during a series of physical activities. Therefore, it is possible to collect highly accurate activity intensity for the physical activity to be analyzed, and it is possible to accurately evaluate the activity content.

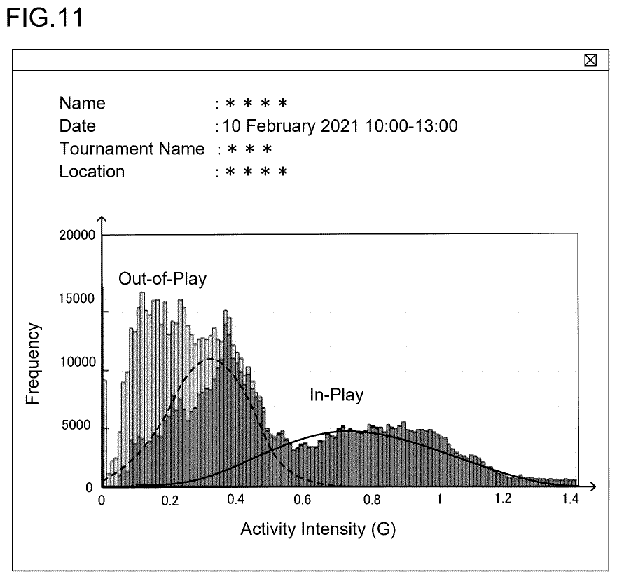

FIG. 11 is an explanatory diagram showing a modified example of the exercise evaluation screen. In the present embodiment, a sequence of physical activity time may be divided into in-play time and out-of-play time. Therefore, in addition to the activity intensity during in-play time, the activity intensity during out-of-play time can also be presented to the user. In this case, in step S41 in the process shown in FIG. 10, the control unit 21 extracts the activity intensity during the in-play time and also extracts the activity intensity during the out-of-play time. Then, in step S15, the control unit 21 calculates a frequency distribution of the activity intensity during the in-play time and a frequency distribution of the activity intensity during the out-of-play time. In addition, in step S17, the control unit 21 generates a histogram showing the frequency distribution of the activity intensity during the in-play time and a histogram showing the frequency distribution of the activity intensity during the out-of-play time. In addition, in step S18, the control unit 21 generates an exercise evaluation screen that displays the histogram showing the frequency distribution of the activity intensity during the in-play time, the histogram showing the frequency distribution of the activity intensity during the out-of-play time, and a curve showing a mixed normal distribution model (two normal distributions) that approximates the histogram of the in-play time. As a result, an exercise evaluation screen shown in FIG. 11 is generated. In addition, in the screen shown in FIG. 11, the histogram of the in-play time is displayed in a darker color in front of the histogram of the out-of-play time. By presenting such a screen, the activity intensity during the in-play time can be evaluated, and the activity intensity during the out-of-play time can also be grasped.

In the present embodiment, the same effects as those in Embodiment 1 described above can be obtained. In addition, in the present embodiment, since acceleration data in a time zone to be analyzed can be extracted from acceleration data measured during a series of physical activities, the content of the physical activity to be analyzed can be evaluated more accurately. For example, in physical activities such as rehabilitation and labor, as well as sports, activities (movements, work) are performed with breaks in between. Even in such physical activities, acceleration data collected using the wearable device 10 can be divided into acceleration data during activities (movements, work) and acceleration data during rest, so that the activity content can be evaluated based on the acceleration data during activity. In addition, the threshold value of the activity intensity for determining whether the user is active or resting in physical activity may be set appropriately according to the content of physical activity. In addition, in the present embodiment as well, the modified examples described appropriately in Embodiment 1 above can be applied.

### Embodiment 3

In Embodiments 1 and 2 described above, the time-series changes in activity intensity in physical activity are expressed by the mixed normal distribution model, and the activity content is evaluated according to the shapes of two normal distributions in the mixed normal distribution model. Here, the inventors have found out that by expressing the time series changes in activity intensity using the mixed normal distribution model and focusing on the normal distribution on the high intensity side of the mixed normal distribution model, the content of physical activity can be evaluated more sensitively.

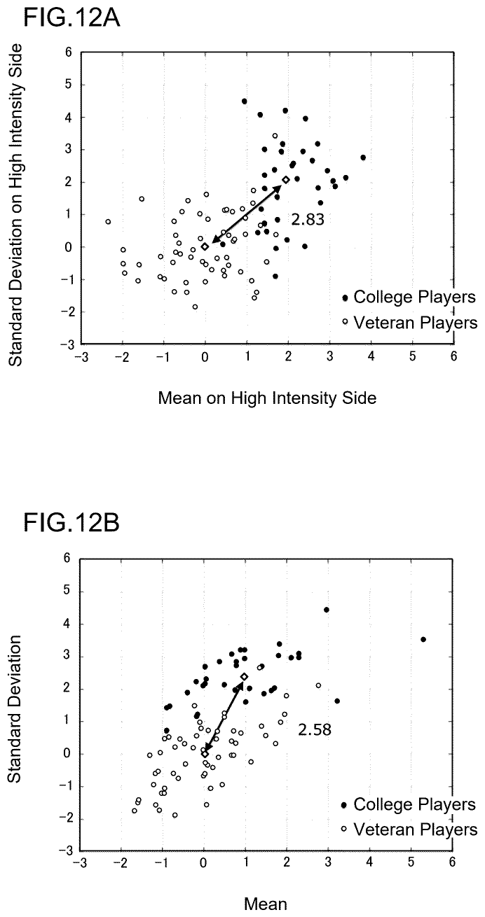

FIGS. 12A and 12B are explanatory diagrams of the process of evaluating the content of physical activity based on the activity intensity on the high intensity side. FIG. 12A shows a chart plotting the mean and standard deviation in a normal distribution on the high intensity side when the activity intensities obtained when a plurality of users perform physical activities are expressed by the mixed normal distribution model. In addition, in the example shown in FIG. 12A, data of the normal distribution on the high intensity side is shown when activity intensities collected during tennis matches for college students in their 20s (college players) and players in their 30s to 60s (veteran players) are fitted to the mixed normal distribution model. In FIG. 12A, data for college students in their 20s is plotted as black circles, and data for players in their 30s to 60s is plotted as white circles. It is predicted that the activity intensity during tennis play will differ significantly between the two groups: college students in their 20s and players in their 30s to 60s. In addition, FIG. 12B shows a chart in which the simple mean and standard deviation of all activity intensities collected during tennis matches similarly for college students in their 20s and players in their 30s to 60s are calculated without fitting the activity intensities to the mixed normal distribution model and the calculated mean and standard deviation are plotted. In FIGS. 12A and 12B, the mean and standard deviation on the high intensity side and the simple mean and standard deviation of all activity intensities are plotted as standardized variables.

In FIG. 12A, two pieces of data plotted as diamonds show the average value of data (mean and standard deviation) of the normal distribution on the high intensity side for college students in their 20s and the average value of data (mean and standard deviation) of the normal distribution on the high intensity side for players in their 30s to 60s. Similarly, in FIG. 12B, two pieces of data plotted as diamonds show the average values of the simple mean and standard deviation of all activity intensities for college students in their 20s and the average values of the simple mean and standard deviation of all activity intensities for players in their 30s to 60s. In the examples shown in FIGS. 12A and 12B, when the statistical distance between the two average values plotted as diamonds was calculated, the statistical distance was 2.83 when the mixed normal distribution model was applied (FIGS. 12A) and 2.58 when the mixed normal distribution model was not applied (FIG. 12B). This means that using the normal distribution data on the high intensity side, which is obtained by applying the mixed normal distribution model to the frequency distribution of activity intensity, can increase the statistical distance between the two population groups. The increased statistical distance means that the characteristics of each group can be more clearly expressed. Therefore, rather than using the simple mean and standard deviation of all activity intensities, it is possible to more accurately express the characteristics of the content of physical activity by using normal distribution data on the high intensity side obtained by applying the mixed normal distribution model.

Therefore, in the present embodiment, by presenting the user with normal distribution data (mean and standard deviation) on the high intensity side when the mixed normal distribution model is applied, it is possible to compare the state of the user's activity intensity with other users.

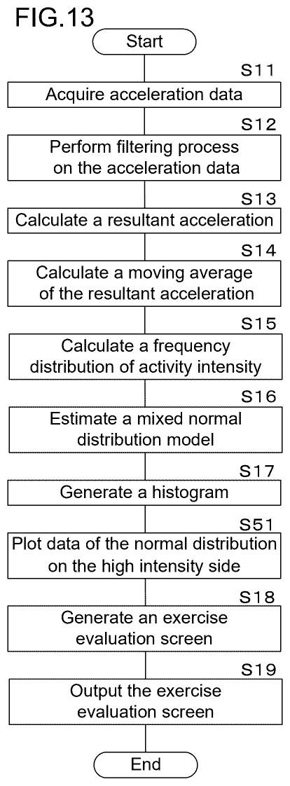

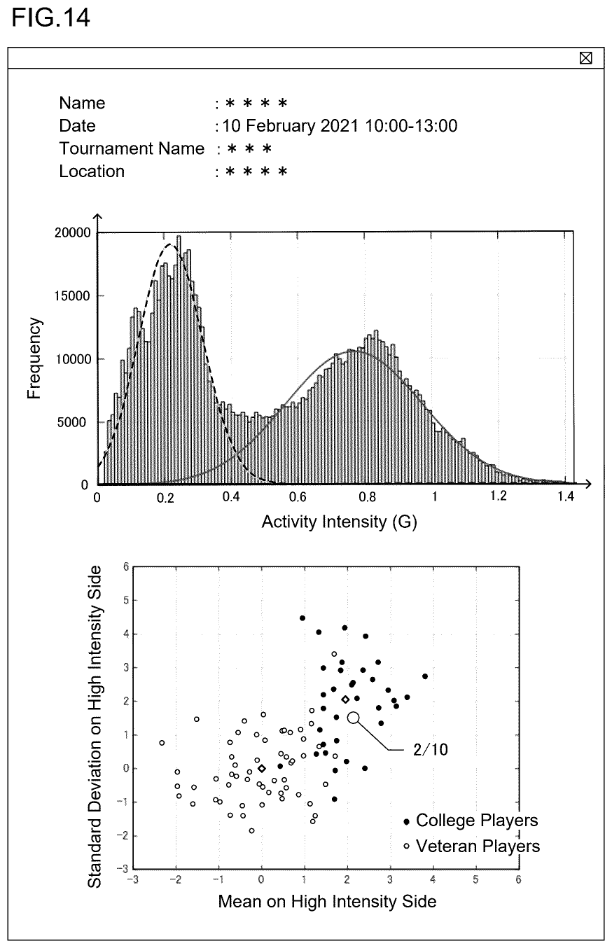

FIG. 13 is a flowchart showing an example of the procedure of an activity intensity determination process according to Embodiment 3, and FIG. 14 is an explanatory diagram showing an example of a screen. The process shown in FIG. 13 is obtained by adding step S51 between steps S17 and S18 in the process shown in FIG. 3. Explanation of the same steps as those in FIG. 3 will be omitted.

In the information processing apparatus 20 according to the present embodiment, the control unit 21 performs the same processing as steps S11 to S17 in FIG. 3. As a result, in the present embodiment as well, the information processing apparatus 20 generates a histogram showing the frequency distribution of activity intensity based on the acceleration data acquired from the wearable device 10, and estimates a mixed normal distribution model that approximates the histogram.

Then, the control unit 21 plots, for the chart shown in FIG. 12A, the data (mean and standard deviation) of the normal distribution on the high intensity side in the mixed normal distribution model estimated in step S16 (S51). In addition, the chart shown in FIG. 12A plots normal distribution data on the high intensity side based on the activity intensities collected for the respective users (here, college students in their 20s and players in their 30s to 60s) of a group set as evaluation criteria in advance, and such a chart may be generated in advance and stored in the storage unit 22. In addition, the chart shown in FIG. 12A may be updated and stored in the storage unit 22 every time the data of users belonging to each group is plotted.

Then, the control unit 21 generates an exercise evaluation screen that displays a histogram, on which a curve showing the mixed normal distribution model is superimposed, and a chart, on which normal distribution data on the high intensity side is plotted (S18). Here, the control unit 21 generates an exercise evaluation screen shown in FIG. 14. The screen shown in FIG. 14 has the same configuration as the screen shown in FIG. 5, and further displays a chart in which the data (mean and standard deviation) of the normal distribution on the high intensity side in the mixed normal distribution model estimated for the activity intensity of the user to be analyzed is plotted in the chart shown in FIG. 12A. In the screen shown in FIG. 14, in the chart in which the normal distribution data on the high intensity side is plotted, the data of the user to be analyzed is shown by a large white circle. In the example shown in FIG. 14, the data of the user to be analyzed is displayed in association with the analysis date (date of physical activity). By presenting such a screen, it is possible to compare the user to be analyzed with each user in the evaluation criteria group for the activity content with high activity intensity. Therefore, it is possible to understand the current status of the user to be analyzed in the evaluation criteria group. For example, in the screen shown in FIG. 14, it can be seen that the user to be analyzed can perform high-intensity activity at the same level as college students in their 20s. Therefore, the mean and standard deviation indicating the distribution on the high intensity side in the mixed normal distribution model specified from the distribution of activity intensities for each player can be output as information regarding the exercise of each player.

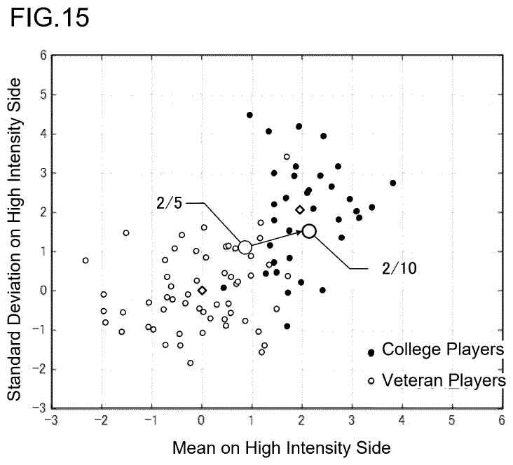

FIG. 15 is an explanatory diagram showing a modified example of the exercise evaluation screen. In the present embodiment as well, when data on the activity intensity of each user's past physical activity is stored in the storage unit 22 or other storage devices, the changes from past data for normal distribution data on the high intensity side can also be presented using the chart shown in FIG. 15. In this case, in step S51 in the process shown in FIG. 13, the control unit 21 plots, for the chart shown in FIG. 12A, the normal distribution data on the high intensity side in the mixed normal distribution model estimated in step S16 and the normal distribution data on the high intensity side in the past physical activity (S51). Therefore, since it is possible to display the chart shown in FIG. 15 on the screen shown in FIG. 14, it is possible to perform comparison with the past physical activity data. In addition, in the chart shown in FIG. 15, an arrow pointing from the past physical activity data to the current physical activity data is displayed. Through this arrow, it is possible to present the change from the past physical activity. In addition, by plotting data on physical activity resulting from different training menus in the chart shown in FIG. 15, it is possible to compare the different physical activity contents resulting from different training menus.

In the present embodiment, the same effects as those in Embodiments 1 and 2 described above can be obtained. In addition, in the present embodiment, the characteristics of the activity content can be evaluated more accurately based on components on the high intensity side of the activity intensity measured during physical activity. In addition, in the present embodiment as well, the modified examples described appropriately in Embodiment 1 above can be applied.

In Embodiments 1 to 3 described above, the frequency distribution of the activity intensity is expressed by a mixed normal distribution model in which two normal distributions, a normal distribution on the high intensity side and a normal distribution on the low intensity side, are mixed. However, the present invention is not limited to this configuration, and the frequency distribution of the activity intensity may be expressed by a mixed normal distribution model in which three or more normal distributions are mixed. With such a configuration, it is possible to analyze each intensity included in the activity content, the amount of activity at each intensity, and the like for physical activity including three or more types of intensities, such as not only high intensity and low intensity but also medium intensity.

In addition, in Embodiments 1 to 3 described above, the characteristics of the activity intensity applied to a person when the person performs physical activity are determined and presented. However, the determination target of activity intensity during physical activity is not limited to persons and may be, for example, animals such as livestock, laboratory animals, and working animals. For example, by attaching an acceleration sensor to livestock to acquire the distribution of activity intensity, it is possible to determine the activity content of the livestock (the amount of exercise, the quality of exercise, and the like), and depending on the activity content, it is possible to detect, for example, the breeding season. In addition, by attaching an acceleration sensor to animals used in animal experiments to acquire the distribution of activity intensity, it is possible to determine the activity content of the experimental animal. For example, it is possible to detect whether or not the animal is performing the predicted activity content. In addition, by attaching an acceleration sensor to a working animal to acquire the distribution of activity intensity, the activity content of the working animal can be determined, and depending on the activity content, it is possible to consider the timing for rest or consider the training that should be given to the working animal. When attaching an acceleration sensor to an animal, it is also preferable to attach the acceleration sensor to a position close to the center of the animal's body (torso). The same processing as in Embodiments 1 to 3 described above can also be performed on acceleration data acquired by an acceleration sensor attached to an animal, and the characteristics of activity intensity (behavior content) in animals can also be determined by the same processing.

It is to be noted that the disclosed embodiment is illustrative and not restrictive in all aspects. The scope of the present invention is defined by the appended claims rather than by the description preceding them, and all changes that fall within metes and bounds of the claims, or equivalence of such metes and bounds thereof are therefore intended to be embraced by the claims.

It is to be noted that, as used herein and in the appended claims, the singular forms “a”, “an”, and “the” include plural referents unless the context clearly dictates otherwise.

1-12. (canceled)

13. A non-transitory computer-readable storage medium storing a program causing a computer to execute processing of:
acquiring acceleration data detected by an acceleration sensor attached to a subject in exercise;
counting a frequency of each acceleration based on the acceleration data; and
specifying a mixed normal distribution model that expresses a frequency distribution for each acceleration.

14. The non-transitory computer-readable storage medium according to claim 13, wherein the program causes the computer to execute processing of outputting information regarding an exercise of the subject based on the specified mixed normal distribution model.

15. The non-transitory computer-readable storage medium according to claim 13, wherein the program causes the computer to execute processing of outputting a curve showing the specified mixed normal distribution model.

16. The non-transitory computer-readable storage medium according to claim 15, wherein the program causes the computer to execute processing of displaying the curve so as to be superimposed on the frequency distribution for the acceleration.

17. The non-transitory computer-readable storage medium according to claim 13, wherein the program causes the computer to execute processing of:
determining whether the subject is exercising or resting based on the acceleration data; and
counting a frequency of each acceleration based on the acceleration data with which it has been determined that the subject is exercising.

18. The non-transitory computer-readable storage medium according to claim 13, wherein the program causes the computer to execute processing of:
specifying a mixed normal distribution model that expresses a frequency distribution for each acceleration, based on the acceleration data detected by the acceleration sensor, for each predetermined exercise time; and
outputting the mixed normal distribution model specified for each exercise time.

19. The non-transitory computer-readable storage medium according to claim 13, wherein the program causes the computer to execute processing of outputting information regarding an exercise of the subject, including a mean and a standard deviation indicating a distribution on a high acceleration side included in the specified mixed normal distribution model.

20. The non-transitory computer-readable storage medium according to claim 13, wherein the program causes the computer to execute processing of:
specifying a mixed normal distribution model that expresses a frequency distribution for each acceleration, for each subject, based on acceleration data detected by an acceleration sensor attached to each of a plurality of subjects; and
outputting the mixed normal distribution model specified for each of the subjects.

21. The non-transitory computer-readable storage medium according to claim 20, wherein the program causes the computer to execute processing of outputting information regarding an exercise of each of the subjects, including a mean and a standard deviation indicating a distribution on a high acceleration side included in the mixed normal distribution model specified for each of the subjects.

22. The non-transitory computer-readable storage medium according to claim 13, wherein the program causes the computer to execute processing of calculating parameters including a mean and a standard deviation in each of a distribution on a high acceleration side and a distribution on a low acceleration side included in the mixed normal distribution model and a mixture ratio of each distribution.

23. An information processing method in which a computer executes processing of:
acquiring acceleration data detected by an acceleration sensor attached to a subject in exercise;
counting a frequency of each acceleration based on the acceleration data; and
specifying a mixed normal distribution model that expresses a frequency distribution for each acceleration.

24. An information processing apparatus comprising a processor, wherein the processor is capable of executing processing of:
acquiring acceleration data detected by an acceleration sensor attached to a subject in exercise;
counting a frequency of each acceleration based on the acceleration data; and
specifying a mixed normal distribution model that expresses a frequency distribution for each acceleration.
